(19) United States
(12) Patent Application Publication
(10) Pub. No.: US 2026/0108746 A1
(43) Pub. Date: Apr. 23, 2026

(54) FIXATION FEATURE FOR IMPLANTABLE MEDICAL DEVICE

(71) Applicant: Medtronic, Inc., Minneapolis, MN (US)

(72) Inventors: Emily S. Paukert, Plymouth, MN (US); Jason D. Hamack, Ramsey, MN (US); Alexander R. Mattson, Edina, MN (US)

(21) Appl. No.: 19/366,598

(22) Filed: Oct. 23, 2025

Related U.S. Application Data
(60) Provisional application No. 63/710,877, filed on Oct. 23, 2024.

(51) Int. Cl. A61N 1/375; A61N 1/05

(52) U.S. Cl. CPC A61N 1/3756; A61N 1/0565; A61N 1/0573; A61N 1/37512; A61N 1/37518

(57) ABSTRACT

A device comprising: an elongated housing extending from a proximal end to a distal end along a longitudinal axis; an elongated body extending distally extending distally from the distal end of the elongated housing and defining a first electrode at or proximate a distal end of the elongated body; and one or more second electrodes disposed at or proximate the distal end of the elongated housing, wherein the one or more second electrodes are configured to contact wall tissue of the chamber without penetrating the wall tissue, wherein the elongated housing defines one or more ramps, each ramp of the one or more ramps extending at least partially around a perimeter of the elongated housing and along the longitudinal axis from a proximal surface to a distal surface, wherein the distal surface of each ramp defines at least a portion of the distal end of the elongated housing.

### RELATED APPLICATIONS

This application claims the benefit of U.S. Provisional Application Serial Number 63/710,877, filed October 23, 2024, the entire contents of which are incorporated herein by reference.

### TECHNICAL FIELD

The disclosure relates to medical devices, and more particularly to fixation mechanisms of medical devices.

### BACKGROUND

Various types of implantable medical devices (IMDs) have been implanted for treating or monitoring one or more conditions of a patient. Such IMDs may be adapted to monitor or treat conditions or functions relating to heart, muscle, nerve, brain, stomach, endocrine organs or other organs and their related functions. Such IMDs may be associated with leads that position electrodes at a desired location or may be leadless with electrodes integrated with and/or attached to the device housing. These IMDs may have the ability to wirelessly transmit data either to another device implanted in the patient or to another instrument located externally of the patient, or both.

A cardiac pacemaker is an IMD configured to deliver cardiac pacing therapy to restore a more normal heart rhythm. Such IMDs sense the electrical activity of the heart, and deliver cardiac pacing based on the sensed electrical activity, via electrodes. Some cardiac pacemakers are implanted a distance from the heart and coupled to one or more leads that intravascularly extend into the heart to position electrodes with respect to cardiac tissue. Some cardiac pacemakers are sized to be completely implanted within one of the chambers of the heart and may include electrodes integrated with or attached to the device housing rather than leads. Some cardiac pacemakers provide dual chamber functionality, by sensing and/or stimulating the activity of both atria and ventricles, or other multi-chamber functionality. A cardiac pacemaker may provide multi-chamber functionality via leads that extend to respective heart chambers, or multiple cardiac pacemakers may provide multi-chamber functionality by being implanted in respective chambers.

### SUMMARY

In general, this disclosure is directed to implantable medical devices (IMDs) configured to sense and deliver electrical signals to tissue of a patient via a plurality of electrodes at or near a distal end of an elongated housing of the IMD. More particularly, this disclosure is directed to IMDs with an electrode disposed at or near the distal end of the elongated housing.

In some examples, a single IMD is implanted in one chamber of a heart of the patient and is able to sense in and/or deliver cardiac pacing to more than one chamber, which may avoid the need for a leaded device or multiple smaller devices to provide such functionality, which may reduce the amount of material implanted within the patient. In some examples, such an implantable medical device includes a distal electrode that is configured to penetrate through wall tissue of the heart chamber in which the device is implanted, and into wall tissue of another heart chamber. In addition to the distal electrode, the device includes a reference electrode and one or more proximal electrodes configured to contact the wall tissue of the heart chamber. The distal electrode may be a helix configured to penetrate tissue of the patient. The distal electrode may be configured to sense in and/or deliver cardiac pacing to one chamber of the heart and the one or more proximal electrodes may be configured to sense in and/or deliver cardiac pacing to another separate chamber of the heart.

In some examples, the IMD may need to be maintained in a substantially similar position and/or orientation relative to the cardiac tissue of the heart, e.g., to deliver consistent and efficacious cardiac pacing to one or more chambers of the heart. In some examples, this disclosure describes a fixation feature disposed on the distal end of the IMD. The fixation feature may be disposed radially inward of the helix of the distal electrode. The fixation feature may contact the wall tissue to inhibit unintended movement of the IMD relative to the wall tissue. The fixation feature may be shaped to facilitate implantation of the distal electrode into the wall tissue and inhibit unintended movement of the distal electrode out of the wall tissue.

In some examples, this disclosure is directed to a device comprising: an elongated housing extending from a proximal end to a distal end along a longitudinal axis, the elongated housing being configured to be implanted wholly within a chamber of a heart; a first electrode extending distally from the distal end of the elongated housing, the first electrode comprising an elongated body defining a helix; a second electrode disposed on the distal end of the elongated housing, wherein the second electrode is configured to contact wall tissue of the chamber without penetrating the wall tissue; and a fixation feature disposed radially inward of the first electrode and extending distally from the distal end of the elongated housing, wherein the fixation feature is configured to contact the wall tissue without penetrating the wall tissue to inhibit unintended movement of the elongated housing relative to the wall tissue.

In some examples, this disclosure is directed to a fixation device comprising: an elongated body extending distally from a distal end of an implantable medical device, the elongated body comprising: a proximal end located at the distal end of the implantable medical device; and a helix extending distally from the proximal end and defining one or more coils, wherein a distal end of the helix is configured to penetrate into tissue of a patient; and a fixation feature disposed radially inward of the helix and extending distally from the distal end of the implantable medical device, wherein the fixation feature is configured to interface with the tissue without penetrating the tissue to inhibit unintended movement of the elongated body relative to the tissue.

In some examples, this disclosure is directed to a method comprising: inserting a device into a chamber of a heart, the device comprising: an elongated housing extending from a proximal end to a distal end along a longitudinal axis; a first electrode extending distally from the distal end of the elongated housing, the first electrode comprising an elongated body defining a helix; a second electrode disposed on the distal end of the elongated housing; and a fixation feature disposed radially inward of the first electrode and extending distally from the distal end of the elongated housing; advancing the first electrode to penetrate wall tissue of the chamber; placing the second electrode and the fixation feature in contact with the wall tissue of the chamber, wherein when the fixation feature contacts the wall tissue, the fixation feature inhibits unintended movement of the device relative to the wall tissue without penetrating the wall tissue; and delivering cardiac pacing from the device to the wall tissue via at least one of the first electrode or the second electrode.

In some examples, this disclosure is directed to a device comprising: an elongated housing extending from a proximal end to a distal end along a longitudinal axis, the elongated housing being configured to be implanted wholly within a chamber of a heart; an electrode extending distally from the distal end of the elongated housing; an elongated body expending distally from the distal end of the elongated housing, the elongated body defining a helix; and a fixation feature disposed radially inward of the first electrode and extending distally from the distal end of the elongated housing, wherein the fixation feature is configured to contact the wall tissue without penetrating the wall tissue to inhibit unintended movement of the elongated housing relative to the wall tissue.

This summary is intended to provide an overview of the subject matter described in this disclosure. It is not intended to provide an exclusive or exhaustive explanation of the methods and systems described in detail within the accompanying drawings and description below.

### BRIEF DESCRIPTION OF DRAWINGS

The details of one or more examples of this disclosure are set forth in the accompanying drawings and the description below. Other features, objects, and advantages of this disclosure will be apparent from the description and drawings, and from the claims.

FIG. 1 is a conceptual diagram illustrating an example device implanted in the heart of a patient, in accordance with one or more aspects of this disclosure.

FIG. 2 is a perspective diagram illustrating an example configuration of the device of FIG. 1.

FIG. 3 is a perspective diagram illustrating an example of the distal end of the device of FIG. 2.

FIG. 4 is a perspective diagram illustrating a cross-sectional view of the example distal end of the device of FIG. 2, the cross-section being taken along a longitudinal axis of the device.

FIG. 5A is a perspective diagram illustrating an example fixation feature of the device of FIG. 2.

FIG. 5B is a perspective diagram illustrating a top-down view of the example fixation feature of FIG. 5A.

FIG. 6 is a block diagram illustrating an example configuration of an example device of any of FIGS. 1-5B.

FIG. 7 is a conceptual diagram of an example device of any of FIGS. 1-6 implanted at a target implant site.

FIG. 8 is a flowchart illustrating an example process for sensing a cardiac electrical signal and delivering cardiac pacing therapy to a heart of a patient via an example device of any of FIGS. 1-7 .

### DETAILED DESCRIPTION

In general, this disclosure is directed to distal end configurations for implantable medical devices (IMDs). More particularly, this disclosure is directed to IMDs having a fixation feature disposed at or around a distal end of an elongated housing of the IMD. The fixation feature may be disposed radially inward of a distal electrode extending from the distal end of the elongated housing. The fixation feature may contact without puncturing tissue of the patient to inhibit unintended rotation of the IMD relative to, e.g., within, the tissue.

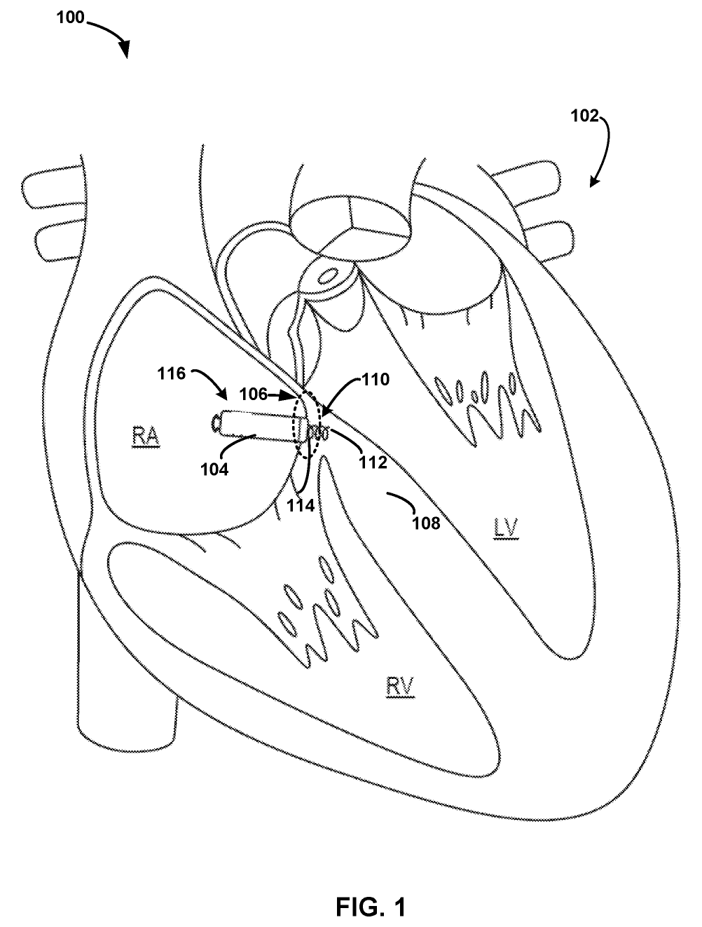

FIG. 1 is a conceptual diagram illustrating an example device 104 implanted in the heart 102 of a patient, in accordance with one or more aspects of this disclosure. Device 104 is shown implanted in the right atrium (RA) of the patient’s heart 102 in a target implant region 106, such as the triangle of Koch, in heart 102 of the patient with a distal end of device 104 directed toward the left ventricle (LV) of the patient’s heart 102. Although in the example of FIG. 1 the distal end of device 104 is directed toward the LV, the distal end may be directed to other targets, such as interventricular septum of heart 102. Target implant region 106 may lie between the bundle of His and the coronary sinus and may be adjacent the tricuspid valve. In some examples, target implant region 106 may be disposed in another position within heart 102, e.g., within a right ventricle (RV) of heart 102.

Device 104 includes a distal end 110 and a proximal end 116. Distal end 110 includes a first electrode 112, and one or more second electrodes 114. First electrode 112 may define a helical shape, e.g., as illustrated in FIG. 1. First electrode 112 extends from distal end 110 and may penetrate through the wall tissue of a first chamber (e.g., the RA in the illustrated example) into wall tissue of a second chamber (e.g., ventricular myocardium 108 of the LV in the illustrated example). Second electrode 114 may include, but is not limited to, a spring electrode, a button electrode, or a cuff electrode (e.g., on an outer perimeter of distal end 110 of device 104). Second electrode 114 may contact the wall tissue of the first chamber as first electrode 112 penetrates the wall tissue of the first chamber. In some examples, when device 104 is affixed to the wall tissue of the first chamber, second electrode 114 at least partially elastically deforms (e.g., compresses) to place an electrically active region of second electrode 114 in contact with the wall tissue (e.g., without puncturing or penetrating the wall tissue). Device 104 may include two or more second electrodes 114 disposed around the outer perimeter of distal end 110, which may allow for delivery of cardiac pacing to tissue at or around target implant region 106 when device 104 is in different orientations and/or around the outer perimeter of distal end 110 of device 104.

In some examples, distal end 110 includes a fixation feature (not shown in FIG. 1) disposed on distal end 110 and radially inward of first electrode 112. The fixation feature may extend distally from distal end 110. The fixation feature may contact wall tissue of heart 102 without puncturing the wall tissue. The fixation feature may interface with the wall tissue to inhibit unintended movement (e.g., unintended rotation) of device 104 relative to the wall tissue (e.g., of first electrode 112 within the wall tissue). The fixation feature may define a pyramidal shape (e.g., a twisted pyramidal shape). The fixation feature may allow for movement (e.g., rotation) of first electrode 112 in a first direction (e.g., into the wall tissue of heart 102) and inhibit movement of first electrode 112 in a second direction opposite the first direction. In some examples, the fixation feature defines a twisted shape (e.g., a twisted pyramidal shape, a twisted plateau shape).

The configuration of electrodes 112 and 114 illustrated in FIG. 1 allows device 104 to sense cardiac signals and/or deliver cardiac pacing to multiple chambers of heart 102, e.g., the RA and ventricle(s) in the illustrated example. In this manner, the configuration of electrodes 112 and 114 may facilitate the delivery of multi-chamber pacing (e.g., DDD pacing) and/or A-V synchronous pacing by single device 104 implanted within the single chamber, e.g., the RA. While device 104 is implanted at target implant region 106 to sense in and/or pace the RA and ventricle(s) in the example shown in FIG. 1, a device having an electrode configuration in accordance with the examples of this disclosure may be implanted at any of a variety of locations to sense in and/or pace any one, two or more chambers of heart 102. For example, device 104 may be implanted at region 106 or another region, and first electrode 112 may extend into tissue, e.g., myocardial tissue, of the LV or interventricular septum to, for example, facilitate the delivery of A-V synchronous pacing. Furthermore, a device having an electrode configuration in accordance with the examples of this disclosure may be implanted at any of a variety of locations within a patient for sensing and/or delivery of therapy to other patient tissue. Such locations include any of the chambers of the heart, in the right atrium at the triangle of Koch or elsewhere therein, in the right ventricle at or near the apex thereof, or in the interventricular septum in the right ventricle, e.g. with the device implanted in the septum so as to enable conduction system pacing (CSP) such as left bundle branch area pacing (LBBAP) with first electrode 112 selectively or non-selectively capturing the left bundle branch (LBB); in such a CSP/LBBAP application (or otherwise), second electrode 114 may optionally be omitted. In some examples, first electrode 112 extends into the tissue of heart 102 at region 106 and affixes device 104 to the tissue of heart 102.

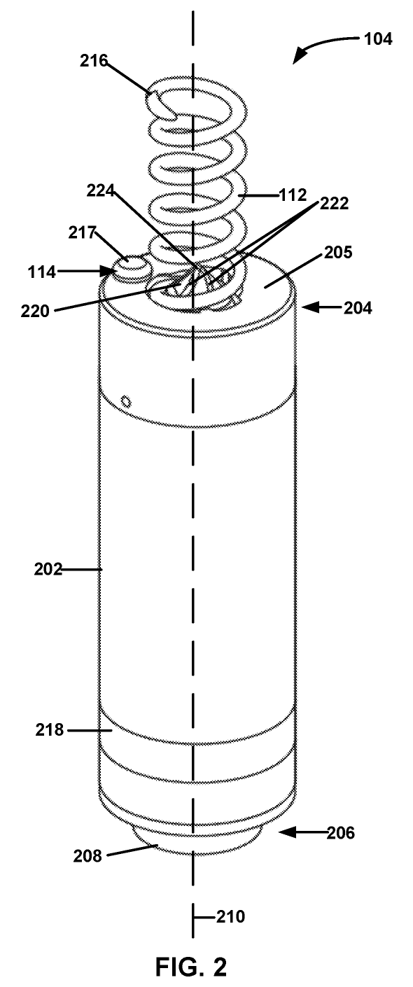

FIG. 2A is a perspective diagram illustrating device 104. Device 104 may include a housing 202 extending from a distal end 204 to a proximal end 206 along longitudinal axis 210. Device 104 may include first electrode 112 extending distally from face 205 of device 104 at distal end 204 of housing 202 and along longitudinal axis 210. Device 104 may further include one or more second electrodes 114 disposed at or around distal end 204 (e.g., on face 205 of device 104). Device 104 may include a fixation feature 220 extending from distal end 204 and disposed radially inward of first electrode 112.

Housing 202 may define a hermetically sealed internal cavity. Housing 202 may be formed from a conductive material including titanium or titanium alloy, stainless steel, MP35N (a non-magnetic nickel-cobalt-chromium-molybdenum alloy), platinum alloy or other bio-compatible metal or metal alloy, or other suitable conductive material. In some examples, housing 202 is formed from a non-conductive material including ceramic, glass, sapphire, silicone, polyurethane, epoxy, acetyl co-polymer plastics, polyether ether ketone (PEEK), a liquid crystal polymer, other biocompatible polymer, or other suitable non-conductive material.

Housing 202 extends between distal end 204 and proximal end 206 along longitudinal axis 210. Housing 202 may be cylindrical or substantially cylindrical but may be other shapes, e.g., prismatic, or other geometric shapes. Housing 202 may include a delivery tool interface member 208, e.g., at proximal end 206, for engaging with a delivery tool during implantation of device 104. At distal end 204, housing 202 may define a face 205 of housing 202. Face 205 may define a distal end major surface. Face 205 may be orthogonal to longitudinal axis 210. In some examples, face 205 is slanted, e.g., face 205 defines a reference plane that is not orthogonal to longitudinal axis 210.

In some examples, an electrode assembly may include first electrode 112 connected to a bushing configured to be affixed to housing 202. The bushing may be at least partially enclosed in coating formed from a polymer or silicon material (e.g., a silicon adhesive). First electrode 112 may extend distally through the coating. The distalmost surface of the coating may define face 205 when the electrode assembly is secured to housing 202. In some examples, as illustrated in FIG. 2, fixation feature 220 may be attached to or may be integral to the bushing. When the bushing is affixed to housing 202, fixation feature 220 may extend distally past distal end 204 along longitudinal axis 210. Fixation feature 220 may at least partially define face 205.

First electrode 112 may extend distally from face 205 along longitudinal axis 210. In some examples, device 104 includes one or more fixation features (e.g., recesses, protrusions, ramps, meshes, tines, or the like) disposed on face 205. First electrode 112 may define a helical or spiral structure. First electrode 112 may extend distally from face 205 to a distal tip. First electrode 112 may define a first electrically active region 216 at or proximate to the distal tip of first electrode 112.

Fixation feature 220 may extend from a proximal base to a distal tip 224 along longitudinal axis 210. Distal tip 224 of fixation feature 220 may be proximal to the distal tip of first electrode 112 and distal to one or more of face 205 or second electrode 114 along longitudinal axis 210. In some examples, as illustrated in FIG. 2, fixation feature 220 includes a plurality of surfaces. The surfaces may define an outer perimeter and an outer surface of fixation feature 220. Circumferentially adjacent surfaces (i.e., surfaces that are next to each other along the outer perimeter of fixation feature 220) may be separated by edges 222. Edges 222 may increase the resistance between fixation feature 220 and the tissue contacting fixation feature 220, thereby inhibiting unintended movement of device 104 in one or more directions when device 104 is implanted in the tissue. Each surface and/or each of edges 222 may be electrically insulated.

Fixation feature 220 may define a polygonal prismatic shape (e.g., a pyramidal shape). Each edge 222 may extend from the proximal base to distal tip 224. Each edge 222 may extend linearly from the proximal base to distal tip 224 or may define one or more curvatures from the proximal base to distal tip 224. In some examples, as illustrated in FIG. 2, fixation feature 220 defines a twisted pyramidal shape, with each edge 222 extending distally along a specific direction of twist. In some examples, fixation feature 220 defines a twisted plateau shape, which may be identical to the twisted pyramidal shape but terminates in a flat surface as opposed to distal tip 224. The direction of twist may be the same as a direction of winding of first electrode 112 (e.g., in a counterclockwise direction), e.g., to allow for implantation of first electrode 112 into the wall tissue and inhibit removal of first electrode 112 out of the wall tissue.

Device 104 may include one or more second electrodes 114 disposed on face 205. Each second electrode 114 may include, but is not limited to, a cuff electrode, a button electrode, a spring electrode, or the like. In some examples where second electrode 114 is a cuff electrode, second electrode 114 may extend from face 205, around an outer edge of face 205, and at least partially along an outer surface of housing 202 defining the outer perimeter of housing 202. Each second electrode 114 may define a second electrically active region 217. Second electrode 114 may be disposed on distal surfaces of ramps extending from face 205. In some examples, second electrode 114 extends distally from or is flush with face 205.

First and second electrodes 112 and 114 may be formed of an electrically conductive material, such as titanium, platinum, iridium, tantalum, stainless steel or alloys thereof. In some examples, second electrode 114 is formed from one or more of Platinum Iridium, a Platinum Iridium-clad alloy (e.g., Platinum Iridium-clad Titanium or Nitinol), Nitinol, or Tantalum Tungsten. First and second electrodes 112 and 114 may be coated with an electrically insulating coating, e.g., a parylene, polyurethane, silicone, epoxy, or other insulating coating, to reduce the electrically conductive active surface area of first and second electrodes 112 and 114, and thereby define first and second electrically active regions 216 and 217. In the example of FIG. 2, first electrically active region 216 includes the distal end of electrode 112. In some examples, first electrically active region 216 is more proximate to the second, e.g., distal end of first electrode 112.

Defining first and second electrically active regions 216 and 217 by covering portions with an insulating coating may increase the electrical impedance of first and second electrodes 112 and 114 and thereby reduce the current delivered during a pacing pulse that captures the cardiac tissue. A lower current drain conserves the power source, e.g., one or more rechargeable or non-rechargeable batteries, of device 104.

Second electrically active regions 217 of the one or more second electrodes 114A may each define a surface area of up to 6 square millimeters (mm²). For example, the surface area of each second electrically active region 217 may be up to 2.5 mm², up to 4.2 mm², or up to 5.8 mm².An example of device 104 with more second electrodes 114 than an otherwise identical device 104 may include second electrodes 114 with reduced surface areas for each second electrically active regions 217 than the other device 104, e.g., such that the total surface area of second electrically active region(s) 217 is independent of the number and/or type of second electrodes 114 on device 104. Second electrodes 114 may be evenly distributed around the outer perimeter of device 104, e.g., to allow for sensing of signals and/or delivery of pacing signals around at least a portion of the outer perimeter of device 104. In some examples, second electrodes 114 are concentrated around a specific portion of the outer perimeter of housing 202.

In the example of FIG. 2, first electrode 112 takes the form of a helix or a coil. First electrode 112 may be an elongated body defining a helix. In some examples, a helix is an object having a three-dimensional shape like that of a wire wound uniformly in a single layer around a cylindrical or conical surface or mandrel such that the wire would be in a straight line if the surface were unrolled into a plane. First electrode 112 may extend from face 205 from a proximal end to a distal end, e.g., defining first electrically active region 216. The proximal end may be a location along first electrode 112 where first electrode 112 extends distally past face 205 of device 104. Fixation feature 220 may be disposed radially inward of first electrode 112. For example, at least a portion of fixation feature 220 may radially and circumferentially overlap with an inner recess defined by first electrode 112.

In some examples, first electrode 112 may be another type of electrode, e.g., a button electrode, a spring electrode, a cuff electrode, a ring electrode, or the like. In such examples, device 104 may include an elongated body defining a fixation helix in addition to first electrode 112. The fixation helix, may be disposed on device 104, e.g., in the same manner as the helix illustrated in FIG. 2. In such examples first electrode 112 may be disposed on (e.g., at or proximate a distal end of), radially inwards of, or radially outwards of the fixation helix.

In some examples, first electrode 112 includes one or more anti-rotation features. The anti-rotation features may facilitate fixation of first electrode 112 to the tissue. The additional anti-rotation features may include a shape of first electrode 112, dimensions (e.g., outer diameter, pitch, or the like) of first electrode 112, one or more features disposed on an outer surface of first electrode 112, or the like. The shape and/or dimensions of first electrode 112 may include a geometric shape of first electrode 112, a varying diameter configuration of first electrode 112, a varying pitch configuration of first electrode 112, a waveform configuration of first electrode 112, or any combination herein. The one or more anti-rotation features disposed on first electrode 112 may include, but are not limited to, elongated darts, barbs, or tines. In some examples, the anti-rotation features include bumps, ridges, recesses, and/or other texturing disposed on face 205. The one or more anti-rotation features may resist rotation of first electrode 112, e.g., by penetrating the tissue, by increasing the friction between first electrode 112 and the tissue, or the like.

First and second electrodes 112 and 114 may vary in size and shape in order to enhance tissue contact of first and second electrically active regions 216 and 217. For example, first electrode 112 may have a round cross-section or could be made with a flatter cross-section (e.g., oval or rectangular) based on tissue contact specifications. In some examples, second electrode 114 defines an outer surface that varies in size and shape (e.g., an oval outer surface, an outer surface with a larger diameter, or the like) in order to enhance tissue contact of second electrically active region 217.

The distal end of first electrode 112 can have a conical, hemi-spherical, or slanted edge distal tip with a narrow tip diameter, e.g., less than 1 millimeter (mm), for penetrating into and through tissue layers. The distal end of first electrode can be a sharpened or angular tip or sharpened or beveled edges, but the degree of sharpness may be constrained to avoid a cutting action that could lead to lateral displacement of the distal end of first electrode 112 and undesired tissue trauma. In some examples, first electrode 112 defines a maximum diameter at its base that interfaces with housing distal end 204. In such examples, the outer diameter of the helix defined by first electrode 112 may decrease from housing distal end 204 to the distal end of first electrode 112. In some examples, the diameter of first electrode 112 varies from a proximal end to the distal end of first electrode 112. The varying diameter may cause first electrode 112 to resist rotation within the tissue of heart 102.

The outer dimensions of first electrode 112 can be substantially straight and cylindrical, with first electrode 112 being rigid in some examples. First electrode 112 may have flexibility in lateral directions, being non-rigid to allow some flexing with heart motion. In a relaxed state, when not subjected to any external forces, first electrode 112 can be configured to maintain a distance between first electrically active region 216 and housing distal end 204.

Distal end of first electrode 112 can pierce through one or more tissue layers to position first electrically active region 216 within a desired tissue layer, e.g., the ventricular myocardium 108 or interventricular septum. Accordingly, first electrode 112 extends a distance from housing distal end 204 corresponding to the expected pacing site depth and may have a relatively high compressive strength along its longitudinal axis, which may be substantially similar to or coincident with longitudinal axis 210, to resist bending in a lateral or radial direction when a longitudinal, axial, and/or rotational force is applied, e.g., to the proximal end 206 of housing 202 to advance device 104 into the tissue at target implant region 106. By resisting bending in a lateral or radial direction, first electrode 112 can maintain a spacing between a plurality of windings of first electrode 112 when first electrode 112 is a helix electrode. The spacing may be a pre-determined pitch of first electrode 112 and may vary from distal end 204 to the distal end of first electrode 112. First electrode 112 may be longitudinally non-compressible. First electrode 112 may also be elastically deformable in lateral or radial directions when subjected to lateral or radial forces, however, to allow temporary flexing, e.g., with tissue motion, but returns to its normally straight position when lateral forces diminish. In some examples, when first electrode 112 is not exposed to any external force, or to only a force along its longitudinal axis (substantially similar to or coincident with longitudinal axis 210), first electrode 112 retains a straight, linear configuration as shown.

As first electrode 112 enters tissue, second electrode(s) 114 may at least partially contact a surface of the tissue. Second electrode(s) 114 may interface with the surface of the tissue without penetrating the surface of the tissue. Second electrode(s) 114 may sense signals from and/or deliver cardiac pacing signals to the tissue surface. In some examples, where second electrode(s) 114 are cuff electrodes as illustrated in FIG. 2A, second electrode(s) 114 may sense signals from and/or deliver cardiac pacing signals to tissue in contact with face 205 and/or with the outer surface of housing 202 extending around distal end 204 of housing 202.

All, substantially all, or a portion of housing 202 may function as an electrode 218, e.g., an anode, during pacing and/or sensing. In some examples, electrode 218 circumscribes a portion of housing 202 at or near proximal end 206. Electrode 218 can fully or partially circumscribe housing 202. FIG. 2 shows electrode 218 extending as a singular band around the outer perimeter of housing 202. Electrode 218 can also include multiple segments spaced a distance apart along a longitudinal axis 210 of housing 202 and/or around a perimeter of housing 202.

When housing 202 is formed from a conductive material, such as a titanium alloy, portions of housing 202 may be electrically insulated by a non-conductive material, such as a coating of parylene, polyurethane, silicone, epoxy or other biocompatible polymer, or other suitable material. For the portions of housing 202 without the non-conductive material, one or more discrete areas of housing 202 with conductive material can be exposed to define electrode 218.

When housing 202 is formed from a non-conductive material, such as a ceramic, glass or polymer material, an electrically-conductive coating or layer, such as a titanium, platinum, stainless steel, alloys thereof, a conductive material may be applied to one or more discrete areas of housing 202 to form electrode 218.

In some examples, electrode 218 is a component, such as a ring electrode, that is mounted or assembled onto housing 202. Electrode 218 may be electrically coupled to internal circuitry of device 104 via electrically-conductive housing 202 or an electrical conductor when housing 202 is a non-conductive material. In some examples, electrode 218 is located proximate to proximal end 206 of housing 202 and can be referred to as a proximal housing-based electrode. Electrode 218 can also be located at other positions along housing 202, e.g., located proximately to distal end 204 or at other positions along longitudinal axis 210.

In some examples, second electrode(s) 114 or electrode 218 is paired with first electrode 112 for sensing ventricular signals and delivering ventricular pacing pulses. In some examples, one or more of second electrodes 114 are paired with electrode 218 or first electrode 112 for sensing atrial signals and delivering pacing pulses to atrial tissue (e.g., to the atrial endocardium) in target implant region 106. In other words, electrode 218 is paired, at different times, with first electrode 112 and/or second electrode 114 for either ventricular or atrial functionality, respectively. In some examples, first and second electrodes 112 and 114 are paired with each other, with different polarities, for atrial and ventricular functionality. In some examples, first electrode 112 is paired with a single second electrode 114 of two or more second electrodes 114. In some examples, first electrode 112 is simultaneously paired with two or more second electrodes 114, e.g., to allow for the sensing of signals from and/or delivery of cardiac pacing signals to tissue at two or more different locations around the outer perimeter of distal end 204 of housing 202.

In some examples, one or more second electrodes 114 are configured as atrial cathode electrodes for delivering pacing pulses to the atrial tissue, e.g., at target implant region 106 in combination with electrode 218. Second electrodes 114 and electrode 218 may also be used to sense atrial P-waves for use in controlling atrial pacing pulses (delivered in the absence of a sensed P-wave) and for controlling atrial-synchronized ventricular pacing pulses delivered using first electrode 112 as a cathode and electrode 218 as the return anode.

A distal end of first electrode 112 can be configured to rest within a ventricular myocardium of the patient, and one or more second electrodes 114 can be configured to contact an atrial endocardium of the patient without penetration of the atrial endocardium. Device 104 may include more or fewer electrodes than two electrodes. In some examples, device 104 includes one or more second electrodes 114 along housing distal end 204. For example, device 104 may include two or three electrodes configured for atrial functionality like second electrode 114, and the three electrodes may be substantially similar or different from one another. Spacing between a plurality of second electrodes 114A may be at an equal or unequal distance. Second electrode(s) 114 may be individually selectively coupled to sensing and/or pacing circuitry enclosed by housing 202 for use as an anode with first electrode 112 or as an atrial cathode electrode, or may be electrically common and not individually selectable. In some examples, in place of first electrode 112, device 104 includes a fixation element (not shown) of similar shape and mechanical properties, but without an electrically active region or electrode formed thereon or borne thereby; in such examples, electrically active region 216 can be positioned on a separate member and/or on the housing 202. In some examples, device 104 only includes first electrode 112 and electrode 218 and does not include any second electrodes 114.

In some examples device 104 includes one or more therapeutic substance dispensing devices, e.g., on face 205. The therapeutic substance dispensing devices may be configured to elute one or more steroids to tissue in proximity to the therapeutic substance dispensing devices over time. The steroid may mitigate inflammation of patient tissue resulting from interaction with device 104. In some examples, the therapeutic substance dispensing devices comprises one or more monolithic controlled release devices (MCRDs). In some examples, the therapeutic substance is disposed over at least a portion of the outer surface of fixation feature 220.

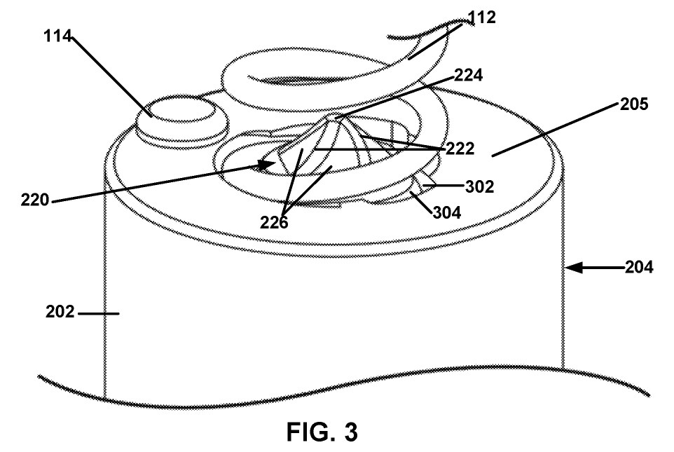

FIG. 3 is a perspective diagram illustrating an example of distal end 204 of device 104 of FIG. 2. First electrode 112 and fixation feature 220 may be affixed to bushing 304 disposed within a recess in housing 202.

Bushing 304 may be permanently or removably attached to housing 202. Bushing 304 may be disposed within a recess in distal end 204 and may interface with feature(s) within the recess to secure bushing 304 to housing 202. The feature(s) may include, but are not limited to, locking tabs, locking recesses, fastener(s), or the like. First electrode 112 may be electrically coupled to components within housing 202 via bushing 304. For example, a feedthrough assembly electrically coupled to first electrode 112 may extending through bushing 304 and into an inner volume of housing 202 containing electrical components of device 104.

Fixation feature 220 may be disposed on top of and/or over a distal surface of bushing 304. Fixation feature 220 may be formed from a biocompatible polymer including, but not limited to, polyether etherketone (PEEK). Fixation feature 220 may extend from a proximal base affixed to bushing 304 to distal tip 224. Fixation feature 220 may include a plurality of surfaces 226 arrayed around an outer perimeter of fixation feature 220. Circumferentially adjacent surfaces 226 around the outer perimeter may be separated by edges 222.

Each of edges 222 may be flush with an adjoining surface 226 or may be proud of the adjoining surface 226. Edges 226 may be rounded, e.g., to reduce tissue irritation when tissue is in contact with fixation feature 220. Edges 222 may extend from the proximal base to distal tip 224. Distal tip 224 may be rounded or otherwise define an atraumatic tip. When device 104 is implanted in the wall tissue of heart 102, distal tip 224 may contact the surface of the wall tissue without puncturing the wall tissue.

Fixation feature 220 may define a twisted shape, where surfaces 226 and edges 222 extend along and around longitudinal axis 210 from the proximal base to distal tip 224. Surfaces 226 and edges 222 may extend around longitudinal axis 210 in a direction of twist. In the example illustrated in FIG. 3, the direction of twist is in the counterclockwise direction, although the direction of twist may be in the clockwise direction in other examples. The direction of twist may be the same as or different from a direction of winding of first electrode 112.

Each edge 222 may extend linearly, along a curve, or along one or more curves from the proximal base to distal tip 224. Each edge 222 may define one or more straight sections and/or one or more curved sections. Each edge 222 may define one or more curves (e.g., two curves separated by a linear portion, two adjoining curves with different radii of curvature) along the length of edge 222. Edges 222 may define a concave shape (e.g., as illustrated in FIG. 3, bowing towards distal end 204 of housing 202) or a convex shape (e.g., bowing away from distal end 204 of housing 202). Edges 222 of fixation feature 220 may all be concave, may all be convex, or may be a mixture of concave and convex.

Proximal end 302 of first electrode 112 may be affixed to bushing 204. A portion of first electrode 112 at or around proximal end 302 may longitudinally overlap within fixation feature 220 along longitudinal axis 210. At least a portion of first electrode 112 may contact fixation feature 220 (e.g., at or around the proximal base of fixation feature 220). In some examples first electrode 112 may be completely separate from fixation feature 220. In some examples, proximal end 302 of first electrode 112 may extend through fixation feature 220 and into bushing 304.

Each of surfaces 226 may include fixation elements disposed over at least a portion of surface 226. Fixation elements may increase fixation of fixation feature 220 to the wall tissue, e.g., without puncturing the wall tissue. Fixation elements may facilitate ingrowth of wall tissue around fixation feature 220. Fixation elements may include, but are not limited to, texturing, protrusions, indentations, or the like.

Fixation feature 220 may be electrically insulated. Surfaces 226 and/or edges 222 may be formed from an electrically insulative material. In some examples, an electrically insulative material may be disposed over an outer surface of fixation feature 220. For example, fixation feature 220 may be formed from a metallic alloy and coated with an electrically insulative material (e.g., parylene). Fixation feature 220 may be formed from a monolithic component or may be formed by affixing two or more sub-components together. Fixation feature 220 may be formed via subtractive manufacturing or via additive manufacturing (e.g., three-dimensional (3D) printing).

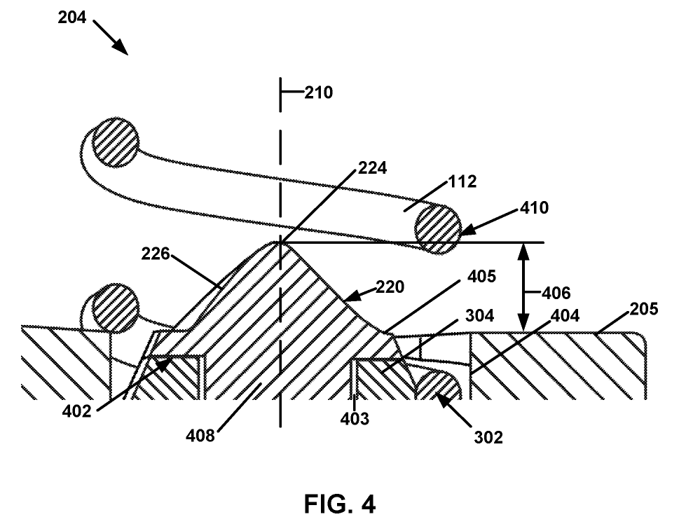

FIG. 4 is a perspective diagram illustrating a cross-sectional view of example distal end 204 of device 104 of FIG. 2, the cross-section being taken along longitudinal axis 210 of device 104. As illustrated in FIG. 3, the cross-section may be taken along a reference plane orthogonal to face 205 of housing 202. Distal end 204 may define recess 404 extending proximally along longitudinal axis 210 from face 205. A distal opening for recess 404 may be flush with face 205. Recess 404 may be sized to retain first electrode 112, bushing 304, and fixation feature 220. For example, an inner diameter of recess 404 may be greater than or equal to a maximum outer diameter for first electrode 112.

First electrode 112 and fixation feature 220 may be affixed to bushing 304. A proximal end 402 of fixation feature 220 may be affixed to and/or in contact with a distal surface of bushing 304, e.g., as illustrated in FIG. 4. Proximal end 402 of fixation feature 220 may be affixed to the distal surface of bushing 304 via welding, soldering, use of an adhesive, use of fastener(s), and/or use of complimentary fixation features on proximal end 402 and on the distal surface of bushing 304. In some examples, as illustrated in FIG. 4, bushing 304 may define an inner recess 403 extending from the distal surface of bushing 304 proximally and/or through bushing 304 along longitudinal axis 210. An extension 408 may be coupled to and extending proximally from proximal end 402 of fixation feature. When fixation feature 220 is coupled to bushing 304, extension 408 may be disposed within inner recess 403, e.g., to facilitate retention of fixation feature 220 to bushing 304.

Surfaces 226 and edges 222 (not pictured in FIG. 4) of fixation feature 220 may extend distally from base 405 of fixation feature 220 to distal tip 224. In some examples, base 405 is the same as proximal end 402 of fixation feature 220. In some examples, as illustrated in FIG. 4, base 405 defines a surface distal to proximal end 402 along longitudinal axis 210. Each surface 226 may extend from a radially outermost position on base 405 (e.g., along the outer perimeter of base 405) or radially inward of the outermost position on base 405. The surface of base 405 may be flush with face 205, proximal to face 205 along longitudinal axis 210, or distal to face 205 along longitudinal axis 210.

Fixation feature 220 may define a height 406 along longitudinal axis 210. Height 406 may be measured as a distance along longitudinal axis 210 from distal tip 224 of fixation feature 220 to face 205 of housing 202. Depending on the position of base 405 relative to face 205, height 406 may be greater than, less than, or the same as a distance along longitudinal axis 210 from distal tip 224 to the surface of base 405. Height 406 may be up to 5 mm (e.g., about 1-2 mm). In some examples, height 406 is about the same as a distance between first electrode 112 and face 205 along longitudinal axis 210 at a position 410 about half a revolution from an exit location of first electrode 112 from face 205.

A starting point of surfaces 226 along base 405 and/or a height 406 of fixation feature 220 may define an offset angle for surfaces 226 (e.g., relative to longitudinal axis 210, relative to face 205) or vice versa. The offset angle may be up to about 75 degrees (e.g., about 20-30 degrees). The offset angle may be selected to allow for sufficient engagement with the wall tissue of heart 102 without puncturing the wall tissue.

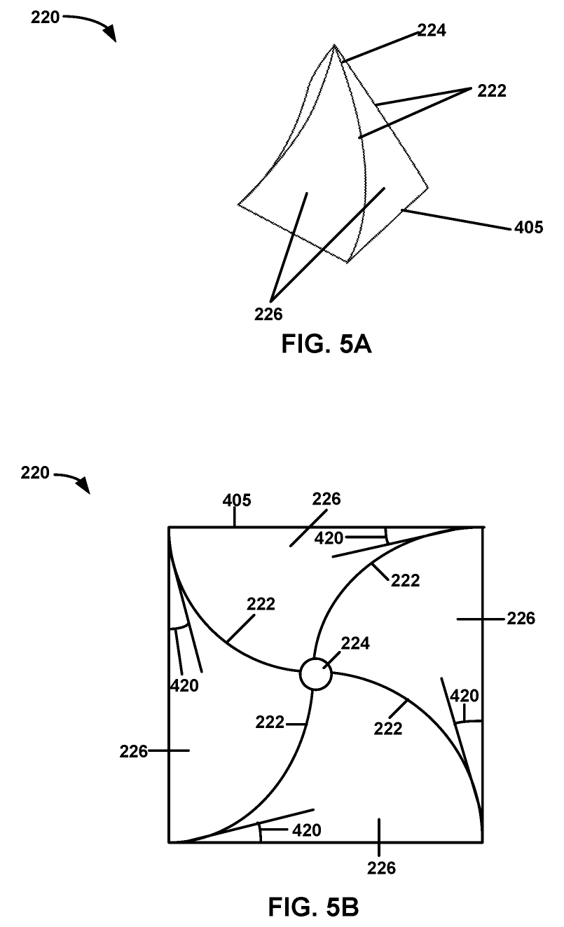

FIG. 5A is a perspective diagram illustrating an example of fixation feature 220 of device 104 of FIG. 2. FIG. 5B is a perspective diagram illustrating a top-down view of example fixation feature 220 of FIG. 5A.

While FIGS. 5A and 5B illustrate fixation feature 220 with four surfaces 226 and four edges 222, other examples may include more or fewer edges 222 and/or surfaces 226. In some examples, as illustrated in FIGS. 5A, and FIG. 5B, surfaces 226 and/or edges 222 are evenly distributed around the outer perimeter of fixation feature 220. In some examples, surfaces 226 and/or edges 222 are biased, e.g., such that a specific portion of the outer perimeter of fixation feature 220 includes fewer or more surfaces 226 and/or edges 222 than another portion of the outer perimeter.

Base 405 may define a circular, triangular, quadrilateral (e.g., as illustrated in FIGS. 5A and 5B), or other polygonal shape. Adjoining features on fixation feature 220 may be referred to as being circumferentially adjacent, although base 405 may not define a circular shape. For example, each edge 222 adjoins two separate surfaces 226, and the two separate surfaces 226 may be referred to herein as circumferentially adjacent surfaces 226.

FIGS. 5A and 5B illustrate an example fixation feature 220 with a twisted shape, e.g., wherein edges 222 and surfaces 226 extend from base 405 to distal tip 224 along and around longitudinal axis 210. The twist of each edge 222 may be define by angle 420. Angle 420 may be measured with reference to an edge of base 405 and/or a reference axis tangential to base 405 (e.g., in the case that base 405 defines a circular shape). Angle 420 may be uniform along the length of edge 222 or may vary along the length of edge 222. For example, as illustrated in FIG. 5B, angle 420 may increase along the length of edge 222 from base 405 towards distal tip 224. In other examples, angle 420 may decrease from base 405 towards distal tip 224 or may alternate between increasing and decreasing from base 405 towards distal tip 224. Angle 420 may be up to 50 degrees (e.g., about 10-45 degrees) at or around base 405. Angle 420 may be up to 90 degrees at or around distal tip 224. At or around distal tip 224.

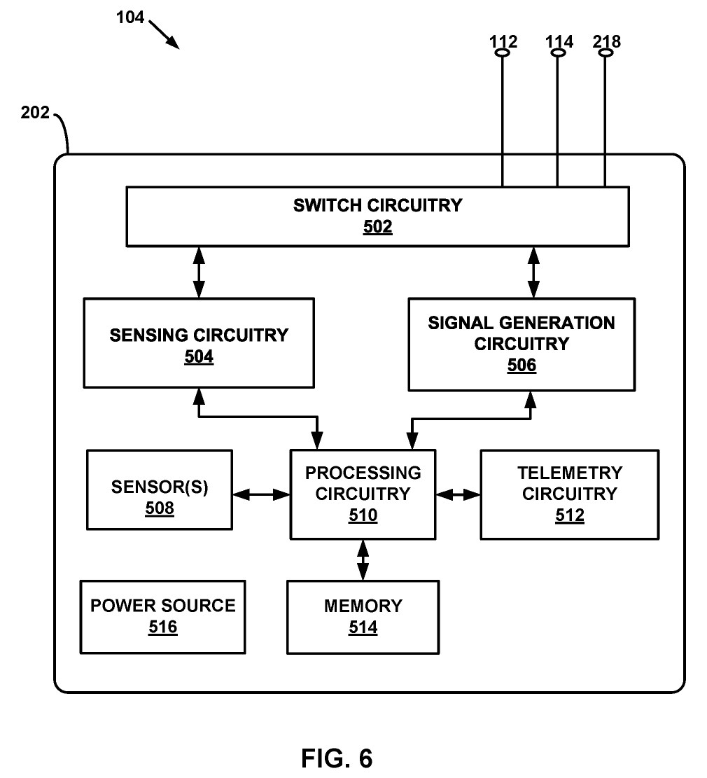

FIG. 6 is a block diagram illustrating an example configuration of an example device 104 of any of FIGS. 1-5B. As illustrated in FIG. 6, device 104 include electrodes 112 and 114, which may be configured as described with respect to FIGS. 1-5B. In the example shown in FIG. 6, device 104 includes switch circuitry 502, sensing circuitry 504, signal generation circuitry 506, sensor(s) 508, processing circuitry 510, telemetry circuitry 512, memory 514, and power source 516. The various circuitry may be, or include, programmable or fixed function circuitry configured to perform the functions attributed to respective circuitry. Memory 514 may store computer-readable instructions that, when executed by processing circuitry 510, cause device 104 to perform various functions. Memory 514 may be a storage device or other non-transitory medium. The components of device 104 illustrated in FIG. 6 may be housed within housing 202.

Signal generation circuitry 506 generates electrical stimulation signals, e.g., cardiac pacing pulses. Switch circuitry 502 is coupled to electrodes 112, 114, and 218 and may include one or more switch arrays, one or more multiplexers, one or more switches (e.g., a switch matrix or other collection of switches), one or more transistors, or other electrical circuitry. Switch circuitry 502 is configured to direct stimulation signals from signal generation circuitry 506 to a selected combination of electrodes 112, 114, and 218, having selected polarities, e.g., to selectively deliver pacing pulses to the RA, ventricles, or interventricular septum of heart 102. For example, in order to pace one or both of the ventricles, switch circuitry 502 may couple first electrode 112, which has penetrated to wall tissue of a ventricle or the intraventricular septum, to signal generation circuitry 506 as a cathode, and one or both of one or more second electrodes 114 or electrode 218 to signal generation circuitry 506 as an anode. As another example, in order to pace the RA, switch circuitry 502 may couple one or more second electrodes 114, which maintains contact with the RA endocardium, to signal generation circuitry 506 as a cathode, and one or both of first electrode 112 or electrode 218 to signal generation circuitry 506 as an anode.

Each of electrodes 112, 114, 218 may be coupled to switch circuitry 502 via a corresponding feedthrough assembly. In some examples, each feedthrough assembly is substantially straight (e.g., along longitudinal axis 210). In some examples, such as when distal end 204 of housing 202 is removable from housing 202 (e.g., when distal end 204 is a removable header), the feedthrough assemblies are offset to allow for removal of distal end 204. For example, when a header defining distal end 204 is configured to be removably secured to housing 202 (e.g., via a turn-lock mechanism), the feedthrough assemblies are offset from longitudinal axis 210 to allow the header to turn relative to housing 202. In some examples, each of electrodes 112, 114, 218 and/or the corresponding feedthrough assemblies are electrically isolated from fixation features 212 on housing 202 of device 104.

Switch circuitry 502 may also selectively couple sensing circuitry 504 to selected combinations of electrodes 112, 114, and 218, e.g., to selectively sense the electrical activity of either the RA or ventricles of heart 102. Sensing circuitry 504 may include filters, amplifiers, analog-to-digital converters, or other circuitry configured to sense cardiac electrical signals via electrodes 112, 114, and/or 218. For example, switch circuitry 502 may couple each of first electrode 112 and second electrode 114 (in combination with electrode 218) to respective sensing channels provided by sensing circuitry 504 to respectively sense either ventricular or atrial cardiac electrical signals. In some examples, sensing circuitry 504 is configured to detect events, e.g., depolarizations, within the cardiac electrical signals, and provide indications thereof to processing circuitry 510. In this manner, processing circuitry 510 may determine the timing of atrial and ventricular depolarizations, and control the delivery of cardiac pacing, e.g., AV synchronized cardiac pacing, based thereon. Processing circuitry 410 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 510 herein may be embodied as firmware, hardware, software or any combination thereof.

Sensor(s) 508 may include one or more sensing elements that transduce patient physiological activity to an electrical signal to sense values of a respective patient parameter. Sensor(s) 508 may include one or more accelerometers, optical sensors, chemical sensors, temperature sensors, pressure sensors, or any other types of sensors. Sensor(s) 508 may output patient parameter values that may be used as feedback to control sensing and delivery of therapy by device 104.

Telemetry circuitry 512 supports wireless communication between device 104 and an external programmer (not shown in FIG. 6) or another computing device under the control of processing circuitry 510. Processing circuitry 510 of device 104 may receive, as updates to operational parameters from the computing device, and provide collected data, e.g., sensed heart activity or other patient parameters, via telemetry circuitry 512. Telemetry circuitry 512 may accomplish communication by radiofrequency (RF) communication techniques, e.g., via an antenna (not shown).

In some examples, where device 104 includes two or more second electrodes 114, processing circuitry 510 may determine a selection of at least one second electrode 114 of the two or more second electrodes 114 for delivery of cardiac pacing signals to heart 102 (e.g., to RA of heart 102) and/or for sensing of signals from heart 102. Processing circuitry 510 may cause switch circuitry 502 to couple the selected at least one second electrode 114 to sensing circuitry 504 and/or to signal generation circuitry 506. Processing circuitry 510 may select the at least one second electrode 114 from the two or more second electrodes 114 in response to communications (e.g., from the external programmer) received by telemetry circuitry 512. In some examples, processing circuitry 510 selects the at least one second electrode 114 based at least in part on one or more signals sensed by device 104 (e.g., via sensing circuitry 504, via sensor(s) 508). The one or more sensed signals may include, but are not limited to, sensed impedance values, pacing capture thresholds, or morphology and/or amplitude signatures of cardiac signals.

Power source 516 delivers operating power to various components of device 104. Power source 516 may include a rechargeable or non-rechargeable battery and a power generation circuit to produce the operating power. Recharging may be accomplished through proximal inductive interaction between an external charger and an inductive charging coil within device 104.

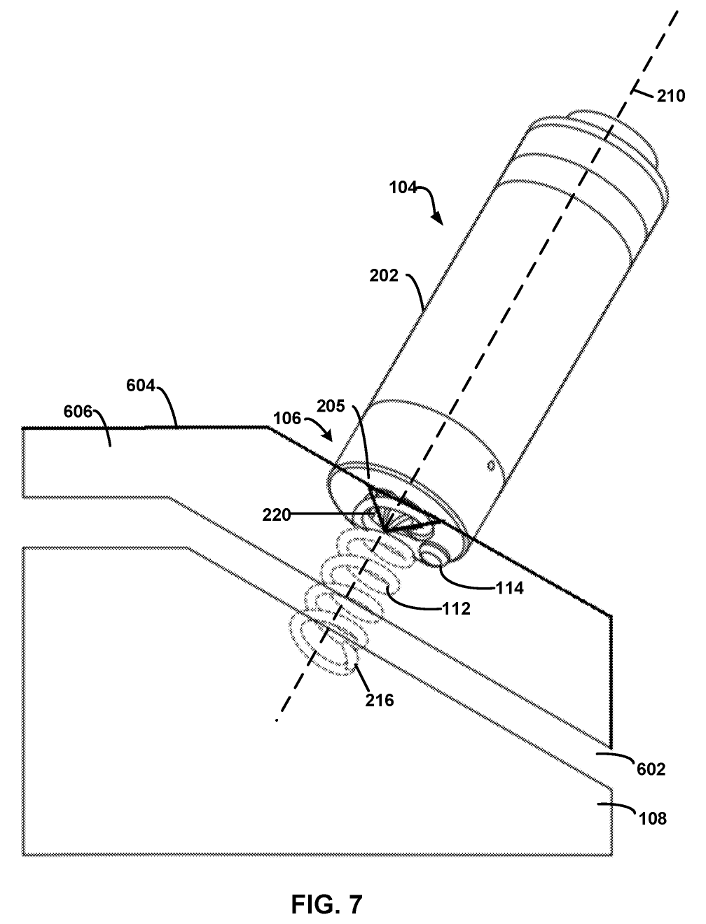

FIG. 7 is a conceptual diagram of device 104 of any of FIGS. 1-6 implanted at one example of a target implant location 106. First electrode 112 may be inserted (e.g., in a manner similar to rotating and advancing a threaded screw) such that tissue becomes engaged with the helix of first electrode 112. As first electrode 112 becomes engaged with tissue, first electrode 112 pierces into the tissue at target implant region 106 and advances through atrial myocardium 606 and central fibrous body 502 to position first electrically active region 216 in ventricular myocardium 108 as shown in FIG. 7. In some examples, first electrode 112 penetrates into the interventricular septum. In some examples, first electrode 112 does not perforate either of the ventricular endocardial or epicardial surface.

In some examples, manual pressure applied to the housing proximal end 206, e.g., via an advancement tool, provides the longitudinal force to pierce the cardiac tissue at target implant region 106. In some examples, actuation of an advancement tool rotates device 104 and first electrode 112 configured as a helix about longitudinal axis 210. The rotation of the helix about the longitudinal axis 210 advances first electrode 112 through atrial myocardium 606 and central fibrous body 602 to position first electrically active region 216 in ventricular myocardium 108 as shown in FIG. 7.

As first electrode 112 advances into the tissue, the distance between second electrode 114 and atrial endocardium 604 decreases until second electrode 114 contact, and may press against, the surface of atrial endocardium 604. In some examples, as first electrode 112 advances into the tissue, fixation feature 220 may contact and abut atrial endocardium 604. Fixation feature 220 may be shaped to facilitate further advancement of first electrode 112 into the tissue. For example, fixation feature 220 may define a twisted feature (e.g., a twisted pyramidal feature) to facilitate further advancement of first electrode 112 into the tissue. Atrial endocardium 604 may interface and apply forces against fixation feature 220 (e.g., against surfaces 226 of fixation feature 226) to inhibit movement and/or rotation of device 104 due to movement of tissue of heart 102 (e.g., movement of ventricular myocardium 108, atrial myocardium 606, central fibrous body 602, or the like) or blood flow during cardiac function. Fixation feature 220 may be shaped to inhibit unintended movement and/or rotation of device 104 to cause device 104 to be retracted from within the tissue. Edges 222 adjoining surfaces 226 may increase the resistance of device 104 to forces acting on device 104. Edges 222 and distal tip 224 of fixation feature 220 may be rounded and/or atraumatic, e.g., to reduce inflammation of tissue and/or a likelihood of puncturing the tissue with fixation feature 220.

Target implant region 106 in some pacing applications is along atrial endocardium 604, substantially inferior to the AV node and bundle of His. For example, target implant region 106 may be at least partially within the Triangle of Koch of heart 102. First electrode 112 can have a length that penetrates through atrial endocardium 604 in target implant region 106, through the central fibrous body 602 and into ventricular myocardium 108 without perforating through the ventricular endocardial surface. In some examples, when the full length of first electrode 112 is fully advanced into target implant region 106, first electrically active region 216 rests within ventricular myocardium 108 and second electrode 114 is positioned in intimate contact with atrial endocardium 604. First electrode 112 may extend from housing distal end 204 approximately 3 mm to 12 mm in various examples. In some examples, first electrode 112 may extend a distance from distal end 204 by at least 3 mm. The diameter of an elongated body defining first electrode 112 may be 4 mm or less, e.g., may be 1 mm or less, may be 0.6 mm or less. An outer diameter of the helix or coil defined by first electrode 112 may be up to 6 mm (e.g., up to 4 mm).

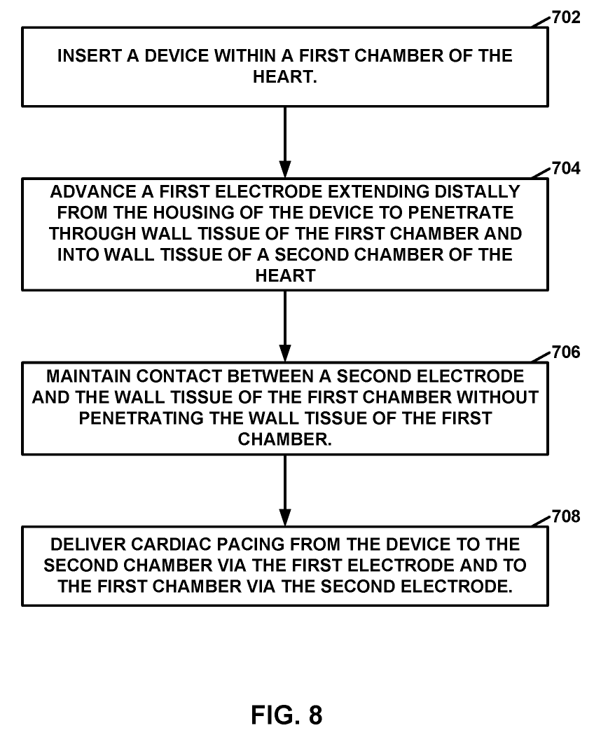

FIG. 8 is a flow diagram illustrating an example process for sensing a cardiac electrical signal and delivering cardiac pacing therapy to heart 102 of a patient via a device 104 of any of FIGS. 1-7. The technique of FIG. 8 will be described with concurrent reference to device 104 as illustrated in FIGS. 1-7, although a person having ordinary skill in the art will understand that the technique may be performed in reference to an implantable medical lead or other medical device.

A clinician may insert device 104 within a single first chamber of the heart 102 (702). The first chamber of heart 102 may be the right atrium, left atrium, the right ventricle, or the left ventricle. The clinician may insert device 104 into the first chamber via delivery tool connected to device 104 (e.g., connected to delivery tool interface member 208). The clinician may advance first electrode 112 extending distally from housing 202 of device 104 to penetrate through wall tissue of the first chamber and into wall tissue of a second chamber of heart 102 (704). In some examples, advancing first electrode 112 includes positioning a distal end of first electrode 112 (e.g., a first electrically active region 216) within a ventricular myocardium 108 of the patient. The clinician may advance first electrode 112 by rotating device 104 clockwise or counterclockwise within the first chamber, depending on how first electrode 112 is wound. Depending on the surface features (e.g., surface curvature) of tissue within the target implantation region 106, the clinician may implant device 104 into the tissue orthogonally or at an angle.

The clinician may cause device 104 to maintain contact between second electrode 114 and the wall tissue of the first chamber, without penetrating the wall tissue of the first chamber (706). The clinician may continue to advance first electrode 112 into the tissue until at least a portion of face 205, second electrode 114, and fixation feature 220 contact the surface of the tissue. Once device 104 is implanted within the wall tissue, portions of the wall tissue may interface with and/or act against surfaces 226 of fixation feature 220, e.g., to inhibit unintended movement and/or rotation of device 104 within the wall tissue. For example, movement within the wall tissue may apply a torque on first electrode 112, and fixation feature 220 may act against the wall tissue to resist rotation of device 104 due to the torque.

Device 104 may deliver cardiac pacing from device 104 to the second chamber via first electrode 112 and to the first chamber via second electrode 114 (708). Device 104 may deliver cardiac pacing to the first chamber and/or the second chamber via first electrode 112, second electrode 114, and/or one or more other electrodes of device 104 (e.g., electrode 218).

It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

In addition, it should be noted that system described herein may not be limited to treatment of a human patient. In alternative examples, the system may be implemented in non-human patients, e.g., primates, canines, equines, pigs, and felines. These other animals may undergo clinical or research therapies that may benefit from the subject matter of this disclosure.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

This disclosure describes each of the following examples.

Example 1: a device comprising: an elongated housing extending from a proximal end to a distal end along a longitudinal axis, the elongated housing being configured to be implanted wholly within a chamber of a heart; a first electrode extending distally from the distal end of the elongated housing, the first electrode comprising an elongated body defining a helix; a second electrode disposed on the distal end of the elongated housing, wherein the second electrode is configured to contact wall tissue of the chamber without penetrating the wall tissue; and a fixation feature disposed radially inward of the first electrode and extending distally from the distal end of the elongated housing, wherein the fixation feature is configured to contact the wall tissue without penetrating the wall tissue to inhibit unintended movement of the elongated housing relative to the wall tissue.

Example 2: the device of example 1, wherein the fixation feature extends distally from a base to a distal tip, and wherein the distal tip defines an atraumatic tip.

Example 3: the device of any of examples 1 or 2, wherein the fixation feature defines a pyramidal shape.

Example 4: the device of example 3, wherein the fixation feature defines a twisted pyramidal shape.

Example 5: the device of example 4, wherein the fixation feature defines a direction of twist along each edge of the twisted pyramidal shape, the direction of twist being the same as a direction of winding of the helix.

Example 6: the device of any of examples 1–5, wherein the fixation feature defines a twisted plateau shape.

Example 7: the device of any of examples 1–6, wherein the helix is configured to penetrate the wall tissue, and wherein the fixation feature is configured to allow the helix to be advanced into the wall tissue in a first direction and to inhibit the helix from being retracted from within the wall tissue in a second direction, the second direction being different from the first direction.

Example 8: the device of any of examples 1–7, wherein the fixation feature defines a plurality of surfaces and a plurality of edges separating circumferentially adjacent surfaces.

Example 9: the device of example 8, wherein each surface of the plurality of surfaces is electrically insulated.

Example 10: the device of any of examples 4, 8, or 9, wherein the fixation feature defines a textured surface along each surface of the plurality of surfaces.

Example 11: the device of any of examples 8–10, wherein each edge of the plurality of edges includes one or more of: a straight section; or a curved section.

Example 12: the device of any of examples 1–11, wherein the chamber of the heart comprises a first chamber of the heart, and wherein the helix is configured to penetrate into wall tissue of a second chamber of the heart that is separated from the first chamber of the heart.

Example 13: the device of example 12, wherein the first chamber of the heart comprises an atrium of the heart, and wherein the second chamber comprises a ventricle of the heart.

Example 14: a fixation device comprising: an elongated body extending distally from a distal end of an implantable medical device, the elongated body comprising: a proximal end located at the distal end of the implantable medical device; and a helix extending distally from the proximal end and defining one or more coils, wherein a distal end of the helix is configured to penetrate into tissue of a patient; and a fixation feature disposed radially inward of the helix and extending distally from the distal end of the implantable medical device, wherein the fixation feature is configured to interface with the tissue without penetrating the tissue to inhibit unintended movement of the elongated body relative to the tissue.

Example 15: the fixation device of example 14, wherein the fixation feature extends distally from a base on the distal end of the implantable medical device to a distal tip, and wherein the distal tip defines an atraumatic tip.

Example 16: the fixation device of any of examples 14 or 15, wherein the fixation feature defines a pyramidal shape.

Example 17: the fixation device of example 16, wherein the fixation feature defines a twisted pyramidal shape.

Example 18: the fixation device of example 17, wherein the fixation feature defines a direction of twist along each edge of the twisted pyramidal shape, the direction of twist being the same as a direction of winding of the helix.

Example 19: the fixation device of any of examples 14–18, wherein the fixation feature is configured to allow the helix to be advanced into the tissue in a first direction and to inhibit the helix from being retracted from within the tissue in a second direction, the second direction being different from the first direction.

Example 20: the fixation device of any of examples 14–19, wherein the fixation feature defines a plurality of surfaces and a plurality of edges separating circumferentially adjacent surfaces.

Example 21: the fixation device of example 20, wherein each surface of the plurality of surfaces is electrically insulated.

Example 22: the fixation device of any of examples 20 or 21, wherein the fixation feature defines a textured surface along each surface of the plurality of surfaces.

Example 23: the device of any of examples 20–22, wherein each edge of the plurality of edges includes one or more of: a straight section; or a curved section.

Example 24: a method comprising: inserting a device into a chamber of a heart, the device comprising: an elongated housing extending from a proximal end to a distal end along a longitudinal axis; a first electrode extending distally from the distal end of the elongated housing, the first electrode comprising an elongated body defining a helix; a second electrode disposed on the distal end of the elongated housing; and a fixation feature disposed radially inward of the first electrode and extending distally from the distal end of the elongated housing; advancing the first electrode to penetrate wall tissue of the chamber; placing the second electrode and the fixation feature in contact with the wall tissue of the chamber, wherein when the fixation feature contacts the wall tissue, the fixation feature inhibits unintended movement of the device relative to the wall tissue without penetrating the wall tissue; and delivering cardiac pacing from the device to the wall tissue via at least one of the first electrode or the second electrode.

Example 25: the method of example 24, wherein the fixation feature extends distally from a base to a distal tip, and wherein the distal tip defines an atraumatic tip.

Example 26: the method of any of examples 24 or 25, wherein the fixation feature defines a pyramidal shape.

Example 27: the method of example 26, wherein the fixation feature defines a twisted pyramidal shape.

Example 28: the method of example 27, wherein the fixation feature defines a direction of twist along each edge of the twisted pyramidal shape, the direction of twist being the same as a direction of winding of the helix.

Example 29: the method of any of examples 24–28, wherein the fixation feature allows the advancing of the first electrode to penetrate the wall tissue in a first direction and inhibits retracting of the first electrode from within the wall tissue in a second direction, the second direction being different from the first direction.

Example 30: the method of any of examples 24–29, wherein the fixation feature defines a plurality of surfaces and a plurality of edges separating circumferentially adjacent surfaces.

Example 31: the method of example 30, wherein each surface of the plurality of surfaces is electrically insulated.

Example 32: the method of any of examples 30 or 31, wherein the fixation feature defines a textured surface along each surface of the plurality of surfaces.

Example 33: the method of any of examples 30–32, wherein each edge of the plurality of edges includes one or more of: a straight section; or a curved section.

Example 34: the method of any of examples 24–33, wherein the chamber of the heart comprises a first chamber of the heart, and wherein advancing the first electrode to penetrate the wall tissue of the chamber comprises advancing the first electrode into wall tissue of a second chamber of the heart that is separated from the first chamber of the heart.

Example 35: the method of example 34, wherein the first chamber of the heart comprises an atrium of the heart, and wherein the second chamber comprises a ventricle of the heart.

Example 36: a device comprising: an elongated housing extending from a proximal end to a distal end along a longitudinal axis, the elongated housing being configured to be implanted wholly within a chamber of a heart; an electrode extending distally from the distal end of the elongated housing; an elongated body expending distally from the distal end of the elongated housing, the elongated body defining a helix; and a fixation feature disposed radially inward of the first electrode and extending distally from the distal end of the elongated housing, wherein the fixation feature is configured to contact the wall tissue without penetrating the wall tissue to inhibit unintended movement of the elongated housing relative to the wall tissue.

Example 37: the device of example 36, wherein the electrode is disposed at or proximate a distal end of the helix.

Example 38 the device of example 36, wherein the helix comprises a fixation helix.

Example 39: the device of any of examples 36–38, wherein the device is configured to deliver conduction system pacing (CSP) signals to the wall tissue via the electrode.

Example 40: the device of example 39, wherein the CSP signals comprises left bundle branch area pacing (LBBAP) signals.

Example 41: the device of any of examples 36–40, wherein the fixation feature extends distally from a base to a distal tip, and wherein the distal tip defines an atraumatic tip.

Example 42: the device of any of examples 36–41, wherein the fixation feature defines a pyramidal shape.

Example 43: the device of example 42, wherein the fixation feature defines a twisted pyramidal shape.

Example 44: the device of example 43, wherein the fixation feature defines a direction of twist along each edge of the twisted pyramidal shape, the direction of twist being the same as a direction of winding of the helix.

Example 45: the device of any of examples 36–44, wherein the fixation feature defines a twisted plateau shape.

Example 46: the device of any of examples 36–45, wherein the helix is configured to penetrate the wall tissue, and wherein the fixation feature is configured to allow the helix to be advanced into the wall tissue in a first direction and to inhibit the helix from being retracted from within the wall tissue in a second direction, the second direction being different from the first direction.

Example 47: the device of any of examples 36–46, wherein the fixation feature defines a plurality of surfaces and a plurality of edges separating circumferentially adjacent surfaces.

Example 48: the device of example 47, wherein each surface of the plurality of surfaces is electrically insulated.

Example 49: the device of any of examples 42, 47, or 48, wherein the fixation feature defines a textured surface along each surface of the plurality of surfaces.

Example 50: the device of any of examples 47–49, wherein each edge of the plurality of edges includes one or more of: a straight section; or a curved section.

Various examples have been described. These and other examples are within the scope of the following claims.

What is claimed is:

1. A device comprising:
an elongated housing extending from a proximal end to a distal end along a longitudinal axis, the elongated housing being configured to be implanted wholly within a chamber of a heart;
a first electrode extending distally from the distal end of the elongated housing, the first electrode comprising an elongated body defining a helix;
a second electrode disposed on the distal end of the elongated housing, wherein the second electrode is configured to contact wall tissue of the chamber without penetrating the wall tissue; and
a fixation feature disposed radially inward of the first electrode and extending distally from the distal end of the elongated housing, wherein the fixation feature is configured to contact the wall tissue without penetrating the wall tissue to inhibit unintended movement of the elongated housing relative to the wall tissue.

2. The device of claim 1, wherein the fixation feature extends distally from a base to a distal tip, and wherein the distal tip defines an atraumatic tip.

3. The device of claim 1, wherein the fixation feature defines a pyramidal shape.

4. The device of claim 3, wherein the fixation feature defines a twisted pyramidal shape.

5. The device of claim 4, wherein the fixation feature defines a direction of twist along each edge of the twisted pyramidal shape, the direction of twist being the same as a direction of winding of the helix.

6. The device of claim 1, wherein the fixation feature defines a twisted plateau shape.

7. The device of claim 1, wherein the helix is configured to penetrate the wall tissue, and wherein the fixation feature is configured to allow the helix to be advanced into the wall tissue in a first direction and to inhibit the helix from being retracted from within the wall tissue in a second direction, the second direction being different from the first direction.

8. The device of claim 1, wherein the fixation feature defines a plurality of surfaces and a plurality of edges separating circumferentially adjacent surfaces.

9. The device of claim 8, wherein each surface of the plurality of surfaces is electrically insulated.

10. The device of claim 8, wherein each edge of the plurality of edges includes one or more of:
a straight section; or
a curved section.

11. A fixation device comprising:
an elongated body extending distally from a distal end of an implantable medical device, the elongated body comprising:
a proximal end located at the distal end of the implantable medical device; and
a helix extending distally from the proximal end and defining one or more coils, wherein a distal end of the helix is configured to penetrate into tissue of a patient; and
a fixation feature disposed radially inward of the helix and extending distally from the distal end of the implantable medical device, wherein the fixation feature is configured to interface with the tissue without penetrating the tissue to inhibit unintended movement of the elongated body relative to the tissue.

12. The fixation device of claim 11, wherein the fixation feature extends distally from a base on the distal end of the implantable medical device to a distal tip, and wherein the distal tip defines an atraumatic tip.

13. The fixation device of claim 11, wherein the fixation feature defines a pyramidal shape.

14. The fixation device of claim 13, wherein the fixation feature defines a twisted pyramidal shape.

15. The fixation device of claim 14, wherein the fixation feature defines a direction of twist along each edge of the twisted pyramidal shape, the direction of twist being the same as a direction of winding of the helix.

16. The fixation device of claim 11, wherein the fixation feature is configured to allow the helix to be advanced into the tissue in a first direction and to inhibit the helix from being retracted from within the tissue in a second direction, the second direction being different from the first direction.

17. A device comprising:
an elongated housing extending from a proximal end to a distal end along a longitudinal axis, the elongated housing being configured to be implanted wholly within a chamber of a heart;
an electrode extending distally from the distal end of the elongated housing;
an elongated body expending distally from the distal end of the elongated housing, the elongated body defining a helix; and
a fixation feature disposed radially inward of the first electrode and extending distally from the distal end of the elongated housing, wherein the fixation feature is configured to contact the wall tissue without penetrating the wall tissue to inhibit unintended movement of the elongated housing relative to the wall tissue.

18. The device of claim 17, wherein the device is configured to deliver conduction system pacing (CSP) signals to the wall tissue via the electrode.

19. The device of claim 18, wherein the CSP signals comprises left bundle branch area pacing (LBBAP) signals.

20. The device of claim 17, wherein the fixation feature defines a twisted pyramidal shape.
